(19) United States
(12) Patent Application Publication
(10) Pub. No.: US 2026/0297558 A1
(43) Pub. Date: Oct. 1, 2026

(54) BIOLOGICAL MATERIAL PURIFICATION METHOD, MAGNETIC BEAD, MAGNETIC BEAD DISPERSION LIQUID, AND BIOLOGICAL MATERIAL PURIFICATION KIT

(71) Applicant: SEIKO EPSON CORPORATION, TOKYO (JP)

(72) Inventors: Soichi MORIYA, SUWA-SHI (JP); Masato HANAMURA, MATSUMOTO-SHI (JP); Yasunari IKEDA, SHIOJIRI-SHI (JP)

(21) Appl. No.: 19/576,081

(22) Filed: Mar. 24, 2026

(30) Foreign Application Priority Data
Mar. 25, 2025 (JP) 2025-050305

(51) Int. Cl. C12N 15/10

(52) U.S. Cl. CPC C12N 15/1013

(57) ABSTRACT

A biological material purification method includes: an adsorption step of mixing magnetic beads and a biological material in a container and adsorbing the biological material to the magnetic beads in the obtained mixed liquid; a separation step of magnetically separating, from the mixed liquid in the container, the magnetic beads to which the biological material is adsorbed; and an elution step of eluting the biological material from the magnetic beads into an elution liquid in the container. The magnetic beads contain a magnetic metal particle and a coating layer containing an inorganic oxide and coating a surface of the magnetic metal particle. The magnetic beads have a particle diameter d31 of 1.1 μm or more and 20.4 μm or less. The magnetic beads have a surface area of 12,000 mm² or more and 230,000 mm² or less.

The present application is based on, and claims priority from JP Application Serial Number 2025-050305, filed Mar. 25, 2025, the disclosure of which is hereby incorporated by reference herein in its entirety.

### BACKGROUND

### 1. Technical Field

The present disclosure relates to a biological material purification method, a magnetic bead, a magnetic bead dispersion liquid, and a biological material purification kit.

### 2. Related Art

JP-A-2017-176023 discloses a nucleic acid-binding solid-phase carrier including magnetic particles of an amorphous metal containing Fe, Cr, Si, and B, and a silicon oxide film provided on surfaces of the magnetic particles.

JP-A-2017-176023 discloses a nucleic acid extraction method including a step of adsorbing a nucleic acid to a nucleic acid-binding solid-phase carrier in an adsorption liquid containing a chaotropic substance, a step of cleaning the nucleic acid-binding solid-phase carrier by disposing, in a wash solution, the nucleic acid-binding solid-phase carrier to which the nucleic acid is adsorbed and vibrating, by a magnetic force, the nucleic acid-binding solid-phase carrier to which the nucleic acid is adsorbed, and a step of eluting the nucleic acid into an elution liquid by disposing, in the elution liquid, the nucleic acid-binding solid-phase carrier to which the nucleic acid is adsorbed.

According to the nucleic acid extraction method described in JP-A-2017-176023, by applying an external magnetic field, the nucleic acid-binding solid-phase carrier to which the nucleic acid is adsorbed can be moved and cleaned, so that the nucleic acid can be efficiently extracted.

JP-A-2017-176023 is an example of the related art.

However, in the nucleic acid extraction method described in JP-A-2017-176023, an adsorption rate and an adsorption amount of the nucleic acid are not sufficient.

On the other hand, the adsorption rate and the adsorption amount of the nucleic acid can be increased by increasing an amount (volume) of the nucleic acid-binding solid-phase carrier to be used, but in that case, the separation of the wash solution is insufficient in the washing step, and an amount of the wash solution (residual liquid) adhering to and remaining on the nucleic acid-binding solid-phase carrier increases.

### SUMMARY

A biological material purification method according to an application example of the present disclosure is a method for purifying a biological material. The method includes: an adsorption step of mixing magnetic beads and the biological material in a container and adsorbing the biological material to the magnetic beads in the obtained mixed liquid; a separation step of magnetically separating, from the mixed liquid in the container, the magnetic beads to which the biological material is adsorbed; and an elution step of eluting the biological material from the magnetic beads into an elution liquid in the container. The magnetic beads contain a magnetic metal particle and a coating layer containing an inorganic oxide and coating a surface of the magnetic metal particle. The magnetic beads in the container have a particle diameter d31 of 1.1 μm or more and 20.4 μm or less. The magnetic beads in the container have a surface area of 12,000 mm² or more and 230,000 mm² or less.

A magnetic bead according to an application example of the present disclosure is a magnetic bead to be used in a biological material purification method, the method including an adsorption step of mixing the magnetic bead and a biological material in a container and adsorbing the biological material to the magnetic bead in the obtained mixed liquid, a separation step of magnetically separating, from the mixed liquid in the container, the magnetic bead to which the biological material is adsorbed, and an elution step of eluting the biological material from the magnetic bead into an elution liquid in the container. The magnetic bead contains: a magnetic metal particle; and a coating layer containing an inorganic oxide and coating a surface of the magnetic metal particle. A particle diameter d31 is 1.1 μm or more and 20.4 μm or less. A surface area when a volume is 7 μL is 12,000 mm² or more and 230,000 mm² or less.

A magnetic bead dispersion liquid according to an application example of the present disclosure contains: the magnetic bead according to the application example of the present disclosure; and a dispersion medium in which the magnetic bead is dispersed.

A biological material purification kit according to an application example of the present disclosure includes: the magnetic bead dispersion liquid according to the application example of the present disclosure.

### BRIEF DESCRIPTION OF THE DRAWINGS

FIG. 1 is a flowchart illustrating a configuration of a biological material purification method according to an embodiment.

FIG. 2 is a schematic diagram illustrating the biological material purification method illustrated in FIG. 1.

FIG. 3 is a schematic diagram illustrating the biological material purification method illustrated in FIG. 1.

FIG. 4 is a schematic diagram illustrating the biological material purification method illustrated in FIG. 1.

FIG. 5 is a cross-sectional view illustrating a magnetic bead according to the embodiment.

FIG. 6 is a cross-sectional view schematically illustrating a biological material purification kit according to the embodiment.

FIG. 7 is Table 1 illustrating conditions for nucleic acid purification using magnetic beads in each Example and evaluation results of the nucleic acid purification.

FIG. 8 is Table 2 illustrating conditions for nucleic acid purification using magnetic beads in each Comparative Example and evaluation results of the nucleic acid purification.

### DESCRIPTION OF EMBODIMENTS

Hereinafter, preferred embodiments of a biological material purification method, a magnetic bead, a magnetic bead dispersion liquid, and a biological material purification kit according to the present disclosure will be described in detail with reference to the accompanying drawings.

The magnetic bead according to the embodiment is a particle group that adsorbs a biological material and is used for magnetic separation. Magnetic separation is a technique of applying an external magnetic field to a container containing a solid phase containing magnetic beads and a liquid phase containing a solvent or the like to magnetically attract the solid phase and thus separating the solid phase from the liquid phase.

Examples of the biological material include a nucleic acid such as DNA, RNA, and hybrids thereof. The biological material may be a biological component other than the nucleic acid. The nucleic acid or the biological component may be present in a state contained in, for example, a biological sample such as a cell or a biological tissue, a virus, or a bacterium. The biological material purification method is a method of purifying such a biological material through steps of, for example, adsorption, separation, washing, and elution.

### 1. BIOLOGICAL MATERIAL PURIFICATION METHOD

Hereinafter, an example of the biological material purification method using magnetic separation will be described. In the following description, a case where the biological material is a nucleic acid will be described as an example.

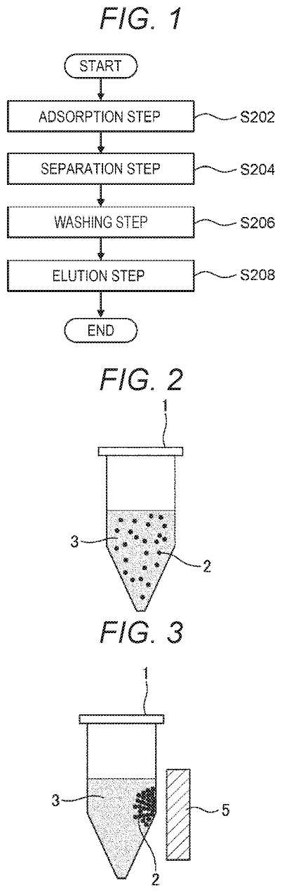

FIG. 1 is a flowchart illustrating a configuration of the biological material purification method according to the embodiment. FIGS. 2 to 4 are schematic diagrams illustrating the biological material purification method illustrated in FIG. 1.

### 1.1. Overview

The biological material purification method illustrated in FIG. 1 is a method for purifying a biological material, and includes an adsorption step S202, a separation step S204, a washing S206, and an elution step S208.

In the adsorption step S202, a mixed liquid obtained by mixing magnetic beads 2, a nucleic acid (biological material) (not illustrated), and a lysis-adsorption liquid (liquid 3 illustrated in FIG. 2) is prepared in a container 1 illustrated in FIG. 2. Then, the nucleic acid is adsorbed to the magnetic beads 2 in the mixed liquid.

In the separation step S204, the magnetic beads 2 to which the nucleic acid is adsorbed are magnetically separated from the mixed liquid in the container 1.

In the washing step S206, the magnetic beads 2 to which the nucleic acid is adsorbed are cleaned with a wash solution in the container 1. In the present disclosure, the washing step S206 is not essential and may be omitted.

In the elution step S208, the nucleic acid is eluted from the magnetic beads 2 to which the nucleic acid is adsorbed in the container 1.

The magnetic beads 2 contain a magnetic metal particle 22 and a coating layer 24 containing an inorganic oxide and coating a surface of the magnetic metal particle 22.

A particle diameter d31 of the magnetic beads 2 in the container 1 is 1.1 μm or more and 20.4 μm or less. Further, a surface area of the magnetic beads 2 in the container 1 is 12,000 mm² or more and 230,000 mm² or less.

According to such a configuration, the particle diameter d31 and the surface area of the magnetic beads 2 are optimized, so that a loss of the magnetic beads 2 during a purification process can be prevented while reducing a time required for adsorbing the biological material. Accordingly, even when an amount of the magnetic beads 2 to be used is reduced, an adsorption amount of the biological material can be secured, and the biological material can be purified with a high yield.

By reducing the amount of the magnetic beads 2 to be used, unintentional remaining of the wash solution or the like between the magnetic beads 2 during the purification process (occurrence of poor purification due to a residual liquid) can be prevented. Accordingly, an inspection failure of the biological material due to residual wash solution being brought in can be prevented.

### 1.2. Adsorption Step

In the adsorption step S202, the lysis-adsorption liquid (liquid 3) to which a specimen sample containing the nucleic acid is added, and the magnetic beads 2 are charged into the container 1 illustrated in FIG. 2. Then, substances accommodated in the container 1 are mixed. Accordingly, as illustrated in FIG. 2, the magnetic beads 2 are dispersed in the lysis-adsorption liquid in the container 1. The nucleic acid is generally enclosed in a cell membrane or a nucleus. Therefore, the nucleic acid is extracted from the specimen sample when a so-called outer shell of the cell membrane or the nucleus is dissolved and removed by a dissolving action of the lysis-adsorption liquid. Thereafter, the nucleic acid is captured by the magnetic beads 2 by an adsorption action of the lysis-adsorption liquid.

As the lysis-adsorption liquid, for example, a liquid containing Proteinase K, a chaotropic substance, and the like is used. Proteinase K degrades proteins and solubilizes nucleic acids. The chaotropic substance generates chaotropic ions in an aqueous solution to reduce an interaction between water molecules, thereby destabilizing a structure. This contributes to the adsorption of the nucleic acid.

As the lysis-adsorption liquid, water such as sterilized water is used. Any additive may be added to the lysis-adsorption liquid. Examples of the additive include a salt, a buffer, a surfactant, an organic solvent, an acid, and a base.

Examples of the salt include sodium chloride, lithium chloride, potassium carbonate, and trisodium citrate.

Examples of the buffer include an acetate buffer, a phosphate buffer, a formic acid buffer, a citrate buffer, and a tartrate buffer.

Examples of the surfactant include a triton-based surfactant such as Triton X-100, a nonionic-based surfactant such as a Tween-based surfactant such as Tween20, and an anionic-based surfactant such as sodium dodecyl sulfate (SDS).

Examples of the organic solvent include alcohols such as ethanol.

FIG. 5 is a cross-sectional view illustrating the magnetic bead 2 according to the embodiment.

The magnetic bead 2 illustrated in FIG. 5 contains the magnetic metal particle 22 and the coating layer 24. A metal powder having magnetism is used as the magnetic metal particle 22. The coating layer 24 contains an inorganic oxide. The magnetic bead 2 will be described in detail later.

### 1.3. Separation Step

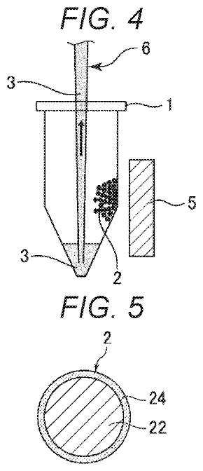

In the separation step S204, an external magnetic field is applied to the mixed liquid containing the magnetic beads 2 to which the nucleic acid is adsorbed, and the magnetic beads 2 are magnetically attracted to an inner wall of the container 1. Accordingly, as illustrated in FIG. 3, the magnetic beads 2 are moved to the inner wall of the container 1 and immobilized. As a result, the magnetic beads 2 as a solid phase and the lysis-adsorption liquid (liquid 3) as a liquid phase can be magnetically separated. In the present specification, the treatment of magnetic separation is referred to as a “magnetic separation treatment”.

Before the magnetic separation treatment is performed, the substances accommodated in the container 1 are stirred as necessary. Accordingly, a probability that the nucleic acid is captured by the magnetic beads 2 can be increased. In the stirring, for example, a vortex mixer, hand shaking, or pipetting is used. This stirring is preferably performed in a state in which no external magnetic field is applied. Accordingly, the magnetic beads 2 are favorably dispersed in the lysis-adsorption liquid, and therefore, the adsorption efficiency can be increased.

For example, a magnet 5 disposed on a side of the container is used to apply the external magnetic field. The magnet 5 may be an electromagnet or a permanent magnet. When the external magnetic field acts on the magnetic beads 2, the magnetic beads 2 move toward the magnet 5.

In a state in which the magnetic beads 2 are immobilized to the inner wall of the container 1 by performing the magnetic separation treatment, as illustrated in FIG. 4, the lysis-adsorption liquid (liquid 3) accumulated at a bottom of the container 1 is sucked and discharged by, for example, a pipette 6 or the like. In the present specification, the treatment of discharging the liquid 3 illustrated in FIG. 4 is referred to as a “liquid discharging treatment”. With the liquid discharging treatment, the magnetic beads 2 capturing the nucleic acid remain in the container 1.

After the liquid discharging treatment is performed, an acceleration may be applied to the container as necessary. Accordingly, the lysis-adsorption liquid adhering to the magnetic beads 2 can be shaken off, and thus the unseparated lysis-adsorption liquid can be reduced. The acceleration may be a centrifugal acceleration. A centrifugal separator may be used to apply the centrifugal acceleration.

### 1.4. Washing Step

In the washing step S206, the magnetic beads 2 to which the nucleic acid is adsorbed are cleaned. Cleaning refers to a treatment of transferring foreign substances captured by the magnetic beads 2 to the wash solution and removing the foreign substances by bringing the magnetic beads 2, to which the nucleic acid is adsorbed, into contact with a wash solution and then separating the magnetic beads 2 from the wash solution again in order to remove the foreign substances.

Specifically, after the wash solution is charged into the container 1 subjected to the separation step S204, the magnetic separation treatment and the liquid discharging treatment described above are performed again. In this case, the liquid 3 in FIGS. 2 to 4 is a wash solution.

In the magnetic separation treatment, first, the magnetic beads 2 and the wash solution in the container 1 are stirred. Accordingly, the magnetic beads 2 are brought into contact with the wash solution and cleaned. At this time, the application of the external magnetic field may be temporarily turned off. Accordingly, the magnetic beads 2 are re-dispersed in the wash solution, and therefore, the cleaning efficiency can be increased. Thereafter, the external magnetic field is applied to immobilize the magnetic beads 2 to the inner wall of the container 1.

In the liquid discharging treatment, the wash solution accumulated on the bottom of the container 1 is discharged in a state in which the magnetic beads 2 are immobilized to the inner wall of the container 1. Accordingly, the magnetic beads 2 are cleaned.

The supply and discharging of the wash solution may be repeated twice or more in total. That is, cleaning may be performed a plurality of times. Accordingly, the foreign substances can be accurately removed.

When the cleaning is performed a plurality of times, the components in the wash solution may be different each time.

The wash solution is not particularly limited as long as the wash solution is a liquid that does not promote elution of the nucleic acid and does not promote binding of the foreign substance to the magnetic beads 2, and examples thereof include organic solvents such as ethanol, isopropyl alcohol, and acetone, aqueous solutions thereof, and low-salt-concentration aqueous solutions, in addition to water.

Various additives may be added to the wash solution. Examples of the additives include buffers such as an acetate buffer and a phosphoric acid buffer, salts such as sodium chloride, and surfactants.

The washing step S206 may be performed as necessary and may be omitted when cleaning is not necessary.

### 1.5. Elution Step

In the elution step S208, the nucleic acid captured by the magnetic beads 2 is eluted into an elution liquid. The elution is a treatment of transferring the nucleic acid to the elution liquid by bringing the magnetic beads 2 to which the nucleic acid is adsorbed into contact with the elution liquid.

Specifically, after the elution liquid is charged into the container 1 containing the magnetic beads 2 to which the nucleic acid is adsorbed, the magnetic separation treatment and the liquid discharging treatment described above are performed again. In this case, the liquid 3 in FIGS. 2 to 4 is an elution liquid.

In the magnetic separation treatment, first, the magnetic beads 2 and the elution liquid in the container 1 are stirred. Accordingly, the elution liquid comes into contact with the magnetic beads 2, and the nucleic acid is eluted into the elution liquid. At this time, the application of the external magnetic field may be temporarily turned off. Accordingly, the magnetic beads 2 are re-dispersed in the elution liquid, and therefore, the elution efficiency can be increased. Thereafter, the external magnetic field is applied to immobilize the magnetic beads 2 to the inner wall of the container 1.

In the liquid discharging treatment, the elution liquid accumulated on the bottom of the container 1 is discharged in a state in which the magnetic beads 2 are immobilized to the inner wall of the container 1. Accordingly, the elution liquid containing the nucleic acid can be collected.

The elution liquid is not particularly limited as long as it is a liquid that promotes elution of the nucleic acid captured by the magnetic beads 2, and examples thereof include water such as sterile water and pure water.

Any additive may be added to the elution liquid. Examples of the additive include a salt, a buffer, a surfactant, an organic solvent, an acid, and a base.

### 1.6. Particle Diameter of Magnetic Beads

Next, the particle diameter d31 of the magnetic beads 2 charged in the container 1 in each of the above steps will be described.

The particle diameter d31 of the magnetic beads 2 charged in the container 1 is 1.1 μm or more and 20.4 μm or less. The particle diameter d31 is preferably 1.5 μm or more and 15.0 μm or less, and more preferably 2.0 μm or more and 8.0 μm or less. When the particle diameter d31 of the magnetic beads 2 is within the above range, the particle diameter d31 of the magnetic beads 2 can be optimized. Accordingly, when a normal amount of the magnetic beads 2 is charged in the container 1, the surface area of the magnetic beads 2 can also be optimized, and thus an occurrence of poor purification due to the residual liquid such as the wash solution can be prevented. The aggregation of the magnetic beads 2 can be prevented, and thus the magnetic beads 2 having excellent redispersibility during stirring can be implemented. Accordingly, a time required for adsorbing the nucleic acid can be sufficiently shortened. Further, when the magnetic separation treatment is performed on the magnetic beads 2, an attractive force and an adsorption force acting on the magnetic beads 2 can be sufficiently secured, and thus the loss of the magnetic beads 2 during the purification process can be prevented.

When the particle diameter d31 of the magnetic beads 2 in the container 1 falls below the above lower limit value, depending on a particle shape of the magnetic beads 2, the surface area of the magnetic beads 2 becomes excessive, and the magnetic beads 2 are likely to aggregate, or the residual liquid such as the wash solution or the like increases. The attractive force and the adsorption force acting on the magnetic beads 2 decrease. On the other hand, when the particle diameter d31 of the magnetic beads 2 exceeds the above upper limit value, depending on the particle shape of the magnetic bead 2, the surface area of the magnetic beads 2 becomes excessively small, and the time required for adsorbing the nucleic acid becomes long, or the adsorption amount decreases. The magnetic beads 2 are likely to settle, an amount of the magnetic beads 2 that can contribute to the adsorption of the nucleic acid may decrease, and the nucleic acid yield may decrease.

The particle diameter d31 of the magnetic beads 2 in the container 1 can be obtained from a cumulative distribution curve obtained from a volume-based particle size distribution measured by a laser diffraction and dispersion method. Specifically, in the cumulative distribution curve, a particle diameter where a cumulative value is 31% from a small diameter side is the particle diameter d31 of the magnetic beads 2. Examples of a device that measures the particle size distribution by using the laser diffraction/scattering method include MT3300 series manufactured by MicrotracBEL Corp.

### 1.7. Surface Area of Magnetic Beads

Next, the surface area C [mm²] of the magnetic beads 2 charged in the container 1 in each of the above steps will be described.

In each of the above steps, the surface area C of the magnetic beads 2 charged in the container 1 is 12,000 mm² or more and 230,000 mm² or less. The surface area C is preferably 15,000 mm² or more and 100,000 mm² or less, and more preferably 20,000 mm² or more and 50,000 mm² or less. By setting the surface area C of the magnetic beads 2 within the above range, the surface area C of the magnetic beads 2 can be optimized. Accordingly, the time required for adsorbing the nucleic acid (nucleic acid adsorption time) can be sufficiently shortened. In the related art, the nucleic acid adsorption time may be shortened by strongly stirring the mixed liquid described above, but in this case, the nucleic acid may be damaged and molecules of the nucleic acid may be cleaved. In contrast, by optimizing the surface area C of the magnetic beads 2, the required adsorption time can be shortened without performing strong stirring. Accordingly, damage to the nucleic acid can be prevented. By setting the surface area C of the magnetic beads 2 within the above range, the occurrence of poor purification due to the residual liquid such as the wash solution can be prevented.

Based on the above, since both the particle diameter d31 and the surface area C of the magnetic beads 2 are optimized, the adsorption amount of the biological material can be secured while reducing the amount of the magnetic beads 2 to be used, the generation of the residual liquid can be prevented, and a biological material purification method capable of satisfactorily purifying the biological material in a high yield can be implemented.

When the surface area C of the magnetic beads 2 in the container 1 falls below the above lower limit value, the adsorption amount of the biological material cannot be secured unless the amount of the magnetic beads 2 to be used is increased. On the other hand, when the surface area C of the magnetic beads 2 in the container 1 exceeds the above upper limit value, depending on the particle shape of the magnetic beads 2, the particle diameter of the magnetic beads 2 becomes excessively small, and thus the magnetic beads 2 are likely to aggregate, the residual liquid such as the wash solution or the like increases, or the attractive force or the adsorption force acting on the magnetic beads 2 decreases.

The surface area C of the magnetic beads 2 in the container 1 is measured using, for example, a BET specific surface area measurement device HM1201-010 manufactured by Mountech Co., Ltd.

### 1.8. Relationship Between Various Parameters of Magnetic Beads and Input Amount of Nucleic Acid

Next, a relationship between various parameters of the magnetic beads 2 and an input amount of the nucleic acid will be described.

### 1.8.1. Required Nucleic Acid Adsorption Time

B is an input amount [μg] of the nucleic acid in the container 1 in the adsorption step S202, and X is an elution amount [μg] of the nucleic acid (nucleic acid elution amount) eluted from the magnetic beads 2 in the elution step S208.

A ratio X/B of the elution amount X to the input amount B is defined as a nucleic acid yield. Further, A is a required nucleic acid adsorption time [min] until the nucleic acid yield X/B exceeds 10%.

At this time, in the biological material purification method according to the embodiment, the required nucleic acid adsorption time A preferably satisfies the following formula (1).

$$A < 9. [ min ] \qquad ( 1 )$$

The required nucleic acid adsorption time A is more preferably 5 min or less, and still more preferably less than 1 min.

According to such a configuration, the time required for implementing a sufficient nucleic acid yield can be sufficiently shortened. Accordingly, the nucleic acid purification can be accelerated.

The nucleic acid yield can be calculated as follows.

First, the elution liquid collected in the elution step S208 is charged in a 1.5 mL tube, and then 80 μL of the elution liquid is taken out from the tube and charged in a spectroscopic cell. Next, 240 μL of pure water is added to the spectroscopic cell to prepare a sample.

Next, the spectroscopic cell containing the sample is set in a cell holder of a spectrophotometer. As the spectrophotometer, a NanoDrop microspectrophotometer manufactured by Thermo Fisher Scientific is used. Then, a nucleic acid concentration of the elution liquid is measured with the spectrophotometer.

Next, a nucleic acid elution amount X [μg] (nucleic acid elution amount) is calculated using the measured nucleic acid concentration and a liquid amount of the elution liquid.

Next, the nucleic acid yield is calculated using the calculated nucleic acid elution amount X [μg] and the nucleic acid input amount B [μg].

### 1.8.2. Relationship Between Surface Area of Magnetic Beads and Input Amount

C is the surface area [mm²] of the magnetic beads 2 in the container 1 in the adsorption step S202.

At this time, a ratio C/B of the surface area C to the input amount B preferably satisfies the following formula (2).

$$1 , 500 [ {mm}^{2} / μ g ] \leq C / B \leq 30 , 000 [ {mm}^{2} / μ g ] \qquad ( 2 )$$

The ratio C/B is more preferably 2,000 [mm²/μg] or more and 20,000 [mm²/μg] or less, and still more preferably 2,500 [mm²/μg] or more and 10,000 [mm²/μg] or less.

According to such a configuration, the surface area C of the magnetic beads 2 can be optimized with respect to the input amount B. Accordingly, the time required for adsorbing the nucleic acid can be sufficiently shortened without performing strong stirring on the mixed liquid. The occurrence of poor purification due to the residual liquid such as the wash solution can be prevented.

When the ratio C/B falls below the above lower limit value, the surface area C of the magnetic beads 2 may be insufficient with respect to the input amount B, and the required nucleic acid adsorption time A may become long or the nucleic acid yield may decrease. On the other hand, when the ratio C/B exceeds the above upper limit value, the surface area C of the magnetic beads 2 may become excessive with respect to the input amount B, and the poor purification due to the residual liquid may occur.

### 1.8.3. Relationship Between Volume of Magnetic Beads and Input Amount

D is a volume [μL] of the magnetic beads 2 in the container 1 in the adsorption step S202.

At this time, a ratio D/B of the volume D to the input amount B preferably satisfies the following formula (3).

$$0.03 [ μ L / μg ] \leq D / B < 0.9 [ μL / μg ] \qquad ( 3 )$$

The ratio D/B is more preferably 0.10 [μL/μg] or more and 0.7 [μL/μg] or less, and still more preferably 0.15 [μL/μg] or more and 0.5 [μL/μg] or less.

According to such a configuration, the volume D of the magnetic beads 2 can be optimized with respect to the input amount B. Accordingly, the volume D of the magnetic beads 2 can be prevented from becoming insufficient or excessive.

When the ratio D/B falls below the above lower limit value, the volume D of the magnetic beads 2 may be insufficient with respect to the input amount B, and the required nucleic acid adsorption time A may become long, or the nucleic acid yield may decrease. On the other hand, when the ratio D/B exceeds the above upper limit value, the volume D of the magnetic beads 2 may become excessive with respect to the input amount B, and the poor purification due to the residual liquid may occur or the loss of the magnetic beads 2 may occur during the purification process.

The volume D of the magnetic beads 2 is a “bulk volume” calculated from a weight F of the magnetic beads 2 and a “loose bulk density” measured using, for example, a powder property evaluation device, Powder Tester (registered trademark) PT-X, manufactured by Hosokawa Micron Corporation.

### 1.8.4. Relationship Between Particle Diameter d31 of Magnetic Beads and Input Amount

E is the particle diameter d31 [μm] of the magnetic beads 2 in the container 1 in the adsorption step S202.

At this time, a ratio E/B of the particle diameter E to the input amount B preferably satisfies the following formula (4).

$$0.13 [ μm / μg ] \leq E / B < 2.8 [ μm / μg ] \qquad ( 4 )$$

The ratio E/B is more preferably 0.25 [μm/μg] or more and 2.0 [μm/μg] or less, and still more preferably 0.35 [μm/μg] or more and 1.0 [μm/μg] or less.

According to such a configuration, the particle diameter E of the magnetic beads 2 can be optimized with respect to the input amount B. Accordingly, depending on the particle shape or the particle diameter d31 of the magnetic beads 2, the surface area C of the magnetic beads 2 can be prevented from becoming insufficient or excessive. The attractive force and the adsorption force of the magnetic beads 2 by the external magnetic field can be optimized.

When the ratio E/B falls below the above lower limit value, depending on the particle shape or the particle diameter d31 of the magnetic beads 2, the surface area C of the magnetic beads 2 may become excessive with respect to the input amount B, and the poor purification due to the residual liquid may occur. As the particle diameter E decreases, the attractive force or the adsorption force of the magnetic beads 2 may be insufficient with respect to the input amount B. On the other hand, when the ratio E/B exceeds the above upper limit value, depending on the particle shape or the particle diameter d31 of the magnetic beads 2, the surface area C of the magnetic beads 2 may be insufficient with respect to the input amount B, and the required nucleic acid adsorption time A may become long or the nucleic acid yield may decrease.

### 1.8.5. Relationship Between Weight of Magnetic Beads and Input Amount

F is the weight [mg] of the magnetic beads 2 in the container 1 in the adsorption step S202.

At this time, a ratio F/B of the weight F to the input amount B preferably satisfies the following formula (5).

$$0.3 [ mg / μg ] \leq F / B < 6.5 [ mg / μg ] \qquad ( 5 )$$

The ratio F/B is more preferably 0.5 [mg/μg] or more and 5.0 [mg/μg] or less, and still more preferably 1.0 [mg/μg] or more and 4.0 [mg/μg] or less.

According to such a configuration, the weight F of the magnetic beads 2 can be optimized with respect to the input amount B. Accordingly, depending on the particle shape or the particle diameter d31 of the magnetic beads 2, the surface area C of the magnetic beads 2 can be prevented from becoming insufficient or excessive.

When the ratio F/B falls below the above lower limit value, depending on the particle shape or the particle diameter d31 of the magnetic beads 2, the surface area C of the magnetic beads 2 may be insufficient with respect to the input amount B. On the other hand, when the ratio F/B exceeds the above upper limit value, depending on the particle shape or the particle diameter d31 of the magnetic beads 2, the surface area C of the magnetic beads 2 may become excessive with respect to the input amount B.

### 2. MAGNETIC BEADS

Next, the magnetic bead 2 (magnetic bead according to the embodiment) will be described.

The magnetic bead 2 illustrated in FIG. 5 is a magnetic bead used in the biological material purification method described above, and contains the magnetic metal particle 22 and the coating layer 24 containing an inorganic oxide and coating the surface of the magnetic metal particle 22.

The particle diameter d31 of the magnetic bead 2 is 1.1 μm or more and 20.4 μm or less, and the surface area when the volume is 7 μL is 12,000 mm² or more and 230,000 mm² or less.

According to such a configuration, the particle diameter d31 and the surface area of the magnetic beads 2 are optimized, and thus the magnetic beads 2 capable of preventing the loss during the purification process while reducing the time required for adsorbing the biological material can be implemented. Accordingly, even when the amount of the magnetic beads 2 to be used is reduced, an adsorption amount of the biological material can be secured, and the biological material can be purified with a high yield.

By reducing the amount of the magnetic beads 2 to be used, unintentional remaining of the wash solution or the like between the magnetic beads 2 during the purification process (occurrence of poor purification due to the residual liquid) can be prevented. Accordingly, the magnetic beads 2 capable of preventing the inspection failure of the biological material due to the residual liquid can be implemented.

The particle diameter d31 of the magnetic beads 2 is preferably 1.5 μm or more and 15.0 μm or less, and more preferably 2.0 μm or more and 8.0 μm or less.

When the particle diameter d31 falls below the above lower limit value, the surface area of the magnetic beads 2 becomes excessive, the magnetic beads 2 are likely to aggregate, and the residual liquid such as the wash solution or the like increases. The attractive force and the adsorption force acting on the magnetic beads 2 decrease. On the other hand, when the particle diameter d31 exceeds the above upper limit value, depending on the particle shape of the magnetic bead 2, the surface area of the magnetic bead 2 becomes excessively small, and the time required for adsorbing the nucleic acid becomes long or the adsorption amount decreases. The magnetic beads 2 are likely to settle, an amount of the magnetic beads 2 that can contribute to the adsorption of the nucleic acid may decrease, and the nucleic acid yield may decrease.

The surface area of the magnetic beads 2 when the volume is 7 μL is preferably 15,000 mm² or more and 100,000 mm² or less, and more preferably 20,000 mm² or more and 50,000 mm² or less.

When the surface area of the magnetic beads 2 when the volume is 7 μL falls below the lower limit value, the adsorption amount of the biological material cannot be secured. On the other hand, when the surface area of the magnetic beads 2 when the volume is 7 μL exceeds the above upper limit value, depending on the particle shape of the magnetic beads 2, the particle diameter of the magnetic beads 2 becomes excessively small, and thus the magnetic beads 2 are likely to aggregate, the residual liquid such as the wash solution or the like increases, or the attractive force or the adsorption force acting on the magnetic beads 2 decreases.

### 2.1. Magnetic Metal Particle

The magnetic metal particle 22 is a metal particle having magnetism.

Examples of a constituent material of the magnetic metal particle 22 include an Fe-based alloy, a Co-based alloy, and an Ni-based alloy. In particular, from the viewpoint of obtaining high saturation magnetization, the constituent material of the magnetic metal particle 22 is preferably an Fe-based alloy (alloy containing Fe as a main component).

The Fe-based alloy may be an alloy having a content of Fe of 50% or more in terms of atomic ratio, and the content of Fe is preferably 70% or more.

The Fe-based alloy contains Fe as a main component, and may contain one or two or more selected from the group consisting of Co, Ni, Cr, Nb, Cu, Al, Mn, Mo, Si, Sn, B, C, P, Ti, and Zr, depending on target characteristics.

An example of the Fe-based alloy is an alloy having a content of Si of preferably 1.0 atomic % or more and 30.0 atomic % or less, more preferably 1.5 atomic % or more and 13.0 atomic % or less, and still more preferably 2.0 atomic % or more and 7.0 atomic % or less. Since such an alloy has a high permeability, the saturation magnetization tends to be high.

The Fe-based alloy may contain at least one of boron (B) having a content of 5.0 atomic % or more and 16.0 atomic % or less and carbon (C) having a content of 0.5 atomic % or more and 5.0 atomic % or less. These elements are elements that promote amorphization, and contribute to stably forming an amorphous structure or a nanocrystal structure in the magnetic metal particle 22.

Further, the Fe-based alloy may contain Cr (chromium) having a content of 1.0 atomic % or more and 8.0 atomic % or less. Accordingly, corrosion resistance of the magnetic metal particle 22 can be improved.

A content of impurities is preferably 1.0 atomic % or less in total. At this level, the above effect exhibited by the magnetic metal particle 22 is less likely to be impaired even when impurities are contained. The impurity in the present specification is an element that is unintentionally mixed in raw materials of the magnetic metal particle 22 or during the production of the magnetic metal particle 22.

A main metal structure forming the magnetic metal particle 22 may take various forms such as a crystal structure, an amorphous structure, and a nanocrystal structure. An amorphous structure refers to a non-crystal structure in which no crystal is present, and a nanocrystal refers to a structure mainly formed of fine crystals having a crystal grain size of 100 nm or less. The amorphous structure and the nanocrystal structure impart a high hardness to the magnetic metal particle 22. When the structure is the amorphous structure or the nanocrystal structure, the coercive force of the magnetic beads 2 has a particularly low value, which contributes to improvement in the redispersibility of the magnetic beads 2. A volume fraction of the magnetic metal particle 22 having an amorphous structure or a nanocrystal structure is preferably 40% or more, and more preferably 60% or more. The volume fraction is obtained based on a result of crystal structure analysis using X-ray diffraction.

The metal structure of the magnetic metal particle 22 can be identified by crystal structure analysis using an X-ray diffraction method on the magnetic metal particle 22. Alternatively, the metal structure can be specified by analyzing a structure observation image or a diffraction pattern obtained with a transmission electron microscope (TEM) from a cut-out sample. For example, in the case of the amorphous structure, a diffraction peak derived from a metal crystal of an α-Fe phase or the like is not observed in peak analysis of the X-ray diffraction method. In the case of the amorphous structure, a so-called halo pattern is formed in an electron diffraction pattern using TEM, and formation of a spot due to a crystal is not observed. The nanocrystal structure is formed of a crystal structure having a grain diameter of, for example, 100 nm or less, and can be checked from a TEM observation image.

The magnetic metal particle 22 particularly preferably contains an Fe-based amorphous alloy. The Fe-based amorphous alloy is an Fe-based alloy including an amorphous structure. Such a magnetic metal particle 22 has a high magnetic permeability and a low coercive force. Therefore, the magnetic bead 2 having good separability in magnetic separation and excellent redispersibility can be obtained by using such a magnetic metal particle 22.

### 2.2. Coating Layer

The coating layer 24 coats the magnetic metal particle 22 and contains an inorganic oxide. According to such a configuration, a function of protecting the magnetic metal particle 22 can be imparted to the coating layer 24. Accordingly, elution of metal ions and the like from the magnetic metal particle 22 can be prevented. As a result, the occurrence of insufficient purification or insufficient inspection due to metal ions or the like can be prevented.

Examples of the inorganic oxide include a silicon oxide, a magnesium oxide, a calcium oxide, an aluminum oxide, a titanium oxide, a zirconium oxide, a boron oxide, a yttrium oxide, and a molybdenum oxide, and a mixture of one or two or more thereof may be used.

The inorganic oxide is preferably a silicon oxide. Since the silicon oxide is chemically stable, oxidation and corrosion of the magnetic metal particle 22 can be particularly prevented, and the corrosion resistance of the magnetic bead 2 can be particularly improved. Good nucleic acid adsorptivity can be imparted to the coating layer 24.

The silicon oxide is represented by a composition formula of SiOₓ (0<x≤2), and is preferably SiO₂. The silicon oxide may form a composite oxide or a composite with one or two or more selected from the group consisting of Al, Ti, V, Nb, Cr, Mn, Sn, and Zr.

The coating layer 24 may contain a substance (impurity) other than the inorganic oxide within a range in which an effect thereof is not impaired, for example, at a ratio of 50 mass % or less of the inorganic oxide described above. When the silicon oxide is used as the inorganic oxide, examples of the impurity include C, N, and P.

A composition of the inorganic oxide can be checked by, for example, EDX analysis or Auger electron spectroscopy.

A thickness of the coating layer 24 is preferably 10 nm or more and 200 nm or less, more preferably 15 nm or more and 100 nm or less, and still more preferably 20 nm or more and 100 nm or less. Accordingly, even when the magnetic beads 2 collide with each other or collide with the inner wall of the container 1 or the like, the coating layer 24 can be prevented from being broken or peeled off. As a result, elution of iron ions and the like due to exposure of the magnetic metal particle 22 can be prevented. A decrease in the magnetization per unit volume of the magnetic beads 2 can be prevented, and a decrease in the movement speed of the magnetic beads 2 can be prevented.

The coating layer 24 may cover a surface of one magnetic metal particle 22, or may cover a plurality of magnetic metal particles 22 together.

The thickness of the coating layer 24 can be measured from, for example, a cross-sectional observation image of the magnetic bead 2 observed with a transmission electron microscope or a scanning electron microscope. Specifically, the thickness of the coating layer 24 can be calculated by acquiring a plurality of cross-sectional observation images of the coating layer 24 and averaging measured values from image processing or the like. For example, the thickness of the coating layer 24 is measured at five or more positions for one magnetic bead 2, an average value is determined, and then the average value is further averaged for ten or more magnetic beads 2.

### 2.3. Characteristics of Magnetic Beads

A saturation magnetization of the magnetic metal particle 22 is preferably 50 emu/g or more, more preferably 80 emu/g or more, and still more preferably 100 emu/g or more. The saturation magnetization is a magnetization value in a case in which a magnetization exhibited by a magnetic material when a sufficiently large magnetic field is externally applied is constant regardless of the magnetic field. When the saturation magnetization is within the above range, a function as a magnetic material can be sufficiently exhibited. Specifically, since a movement speed of the magnetic bead 2 in a magnetic field can be increased, a time required for magnetic separation can be shortened. The saturation magnetization of the magnetic metal particle 22 affects an adsorption force when being fixed by an external magnetic field. When the saturation magnetization is within the above range, a sufficiently high adsorption force can be obtained, and therefore, when the liquid 3 is discharged in a state in which the magnetic beads 2 are immobilized, the magnetic beads 2 can be prevented from being discharged together with the liquid 3. Accordingly, a decrease in nucleic acid yield due to a decrease in the magnetic beads 2 can be prevented.

An upper limit value of the saturation magnetization of the magnetic metal particle 22 is not particularly limited, and is preferably 220 emu/g or less from the viewpoint of ease of selection of a material suitable for a balance between performance and cost.

The saturation magnetization of the magnetic metal particle 22 can be measured by a vibrating sample magnetometer (VSM) or the like. As the vibrating sample magnetometer, for example, TM-VSM1230-MHHL manufactured by TAMAKAWA CO., LTD may be used. A maximum applied magnetic field when measuring the saturation magnetization is, for example, 0.5 T or more.

A coercive force Hc of the magnetic metal particle 22 is preferably 100 A/m or less, more preferably 80 A/m or less, still more preferably 60 A/m or less, and particularly preferably 50 A/m or less. The coercive force Hc refers to a value of an external magnetic field in an opposite orientation required to return a magnetized magnetic material to an unmagnetized state. That is, the coercive force Hc means a resistance force against an external magnetic field. As the coercive force Hc of the magnetic metal particle 22 decreases, the magnetic beads 2 are less likely to aggregate when being switched from a state in which a magnetic field is applied to a state in which no magnetic field is applied, and the magnetic beads 2 can be uniformly dispersed in the liquid 3. Further, even when the switching of the magnetic field application is repeated, the redispersibility of the magnetic beads 2 can be improved. A lower limit of the coercive force Hc of the magnetic metal particle 22 is not particularly limited, and is preferably 5 A/m or more from the viewpoint of ease of selection of a material suitable for a balance between performance and cost.

The coercive force Hc of the magnetic metal particle 22 can be measured by a vibrating sample magnetometer or the like in the same manner as the saturation magnetization described above. A maximum applied magnetic field when measuring the coercive force Hc is, for example, 15 kOe.

### 2.4. Method for Producing Magnetic Beads

Next, an example of a method of producing the magnetic beads 2 will be described.

First, the magnetic metal particles 22 are prepared. The magnetic metal particles 22 are produced using a method according to a general metal powder production method. Examples of the production method include a melting process in which a metal is melted, solidified, and powdered, a chemical process in which a powder is produced by a reduction method or a carbonyl method, and a mechanical process in which a metal having a larger shape such as an ingot is mechanically pulverized to obtain a powder. Among them, a melting process such as an atomization method is suitable for producing the magnetic metal particles 22.

Next, the coating layer 24 is formed on the surface of the magnetic metal particle 22. Examples of a method for forming the coating layer 24 include a wet formation method such as a sol-gel method and a plating method and a dry formation method such as a vapor-phase deposition method. Among them, a Stöber method, which is a type of the sol-gel method, or an atomic layer deposition (ALD) method may be preferably used.

As described above, the magnetic beads 2 containing the magnetic metal particle 22 and the coating layer 24 are obtained.

### 3. BIOLOGICAL MATERIAL PURIFICATION KIT

Next, a biological material purification kit according to the embodiment will be described.

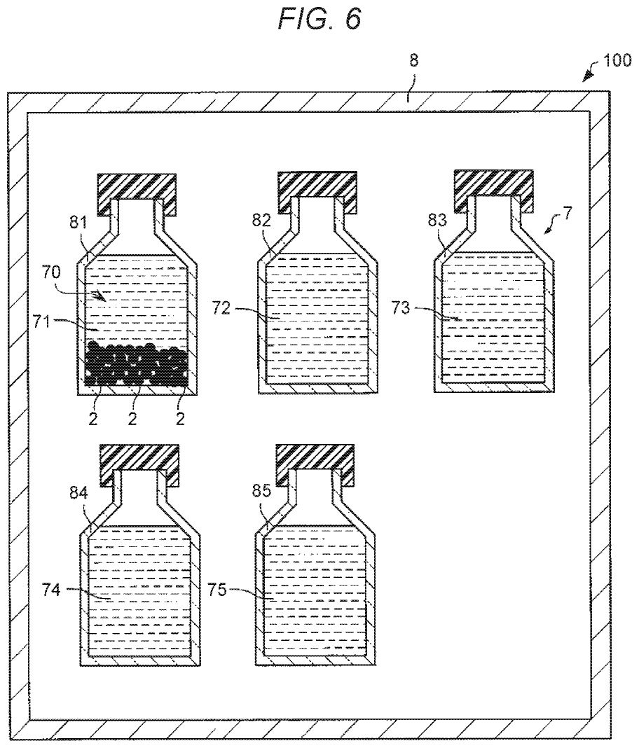

FIG. 6 is a cross-sectional view schematically illustrating a biological material purification kit 100 according to the embodiment.

The biological material purification kit 100 illustrated in FIG. 6 includes a biological material purification reagent set 7 and a housing 8 that accommodates the biological material purification reagent set 7.

The biological material purification reagent set 7 includes a magnetic bead dispersion liquid 70 (magnetic bead dispersion liquid according to the embodiment), a nucleic acid adsorption reagent 72, a first cleaning reagent 73, a second cleaning reagent 74, and a nucleic acid elution reagent 75.

According to such a configuration, the biological material purification kit 100 useful for the biological material purification method described above is obtained.

The magnetic bead dispersion liquid 70 is accommodated in a reagent container 81. The magnetic bead dispersion liquid 70 contains the magnetic beads 2 and a dispersion medium 71 in which the magnetic beads 2 are dispersed. Examples of the dispersion medium 71 include water such as sterilized water. Various additives may be added to the dispersion medium 71. Examples of the additive include the above-described salt, a buffer, and an organic solvent.

The nucleic acid adsorption reagent 72 is accommodated in a reagent container 82. The nucleic acid adsorption reagent 72 contains, for example, a lysis-adsorption liquid and an additive.

The first cleaning reagent 73 is accommodated in a reagent container 83. The first cleaning reagent 73 contains, for example, a first wash solution and an additive.

The second cleaning reagent 74 is accommodated in a reagent container 84. The second cleaning reagent 74 contains, for example, a second wash solution and an additive. The components of the second wash solution are different from those of the first wash solution.

The nucleic acid elution reagent 75 is accommodated in a reagent container 85. The nucleic acid elution reagent 75 contains, for example, an elution liquid and an additive.

The housing 8 has, for example, a bottomed box shape. A shape and a material of the housing 8 are not particularly limited as long as the biological material purification reagent set 7 can be accommodated therein.

The biological material purification kit 100 may contain at least the magnetic bead dispersion liquid 70, and at least one of the nucleic acid adsorption reagent 72, the first cleaning reagent 73, the second cleaning reagent 74, the nucleic acid elution reagent 75, and the housing 8 may be omitted.

### 4. EFFECTS OF EMBODIMENT

As described above, the biological material purification method according to the embodiment is a method for purifying a biological material, and includes the adsorption step S202, the separation step S204, and the elution step S208. In the adsorption step S202, the magnetic beads 2 and the biological material are mixed in the container 1, and the biological material is adsorbed to the magnetic beads 2 in the obtained mixed liquid. In the separation step S204, the magnetic beads 2 to which the biological material is adsorbed are magnetically separated from the mixed liquid in the container 1. In the elution step S208, the biological material is eluted from the magnetic beads 2 into the elution liquid in the container 1.

The magnetic beads 2 contain the magnetic metal particle 22 and the coating layer 24 containing the inorganic oxide and coating the surface of the magnetic metal particle 22.

Further, the particle diameter d31 of the magnetic beads 2 in the container 1 is 1.1 μm or more and 20.4 μm or less, and the surface area C of the magnetic beads 2 in the container 1 is 12,000 mm² or more and 230,000 mm² or less.

According to such a configuration, a biological material purification method capable of satisfactorily purifying a biological material in a high yield can be implemented by securing the adsorption amount of the biological material while reducing the amount of the magnetic beads 2 to be used and preventing the generation of the residual liquid.

In the biological material purification method according to the embodiment, the biological material may be a nucleic acid.

According to such a configuration, the nucleic acid can be efficiently purified.

In the biological material purification method according to the embodiment, the following formula (1) is preferably satisfied,

$$A < 9. [ min ] \qquad ( 1 )$$

where B is the input amount [μg] of the biological material in the container 1, X is the elution amount [μg] of the biological material eluted from the magnetic beads 2 in the elution step S208, and A is the required biological material adsorption time [min] until the ratio X/B of the elution amount X to the input amount B exceeds 10%.

According to such a configuration, the time required for implementing a sufficient nucleic acid yield can be sufficiently shortened. Accordingly, the nucleic acid purification can be accelerated.

In the biological material purification method according to the embodiment, in the adsorption step S202, the following formula (2) is preferably satisfied,

$$1 , 500 [ {mm}^{2} / μ g ] \leq C / B \leq 30 , 000 [ {mm}^{2} / μ g ] \qquad ( 2 )$$

where B is the input amount [μg] of the biological material in the container 1 and C is the surface area [mm²] of the magnetic beads 2 in the container 1.

According to such a configuration, the surface area C of the magnetic beads 2 can be optimized with respect to the input amount B. Accordingly, the time required for adsorbing the nucleic acid can be sufficiently shortened without performing strong stirring on the mixed liquid. The occurrence of poor purification due to the residual liquid such as the wash solution can be prevented.

In the biological material purification method according to the embodiment, in the adsorption step S202, the following formula (3) is preferably satisfied,

$$0.03 [ μ L / μg ] \leq D / B < 0.9 [ μL / μg ] \qquad ( 3 )$$

where B is the input amount [μg] of the biological material in the container 1 and D is the volume [μL] of the magnetic beads 2 in the container 1.

According to such a configuration, the volume D of the magnetic beads 2 can be optimized with respect to the input amount B. Accordingly, the volume D of the magnetic beads 2 can be prevented from becoming insufficient or excessive.

In the biological material purification method according to the embodiment, in the adsorption step S202, the following formula (4) is preferably satisfied,

$$0.13 [ μm / μg ] \leq E / B < 2.8 [ μm / μg ] \qquad ( 4 )$$

where B is the input amount [μg] of the biological material in the container 1 and E is the particle diameter d31 [μm] of the magnetic beads 2 in the container 1.

According to such a configuration, the particle diameter E of the magnetic beads 2 can be optimized with respect to the input amount B. Accordingly, depending on the particle shape or the particle diameter d31 of the magnetic beads 2, the surface area C of the magnetic beads 2 can be prevented from becoming insufficient or excessive. The attractive force and the adsorption force of the magnetic beads 2 by the external magnetic field can be optimized.

In the biological material purification method according to the embodiment, in the adsorption step S202, the following formula (5) is preferably satisfied,

$$0.3 [ mg / μg ] \leq F / B < 6.5 [ mg / μg ] \qquad ( 5 )$$

where B is the input amount [μg] of the biological material in the container 1 and F is the weight [mg] of the magnetic beads 2 in the container 1.

According to such a configuration, the weight F of the magnetic beads 2 can be optimized with respect to the input amount B. Accordingly, depending on the particle shape or the particle diameter d31 of the magnetic beads 2, the surface area C of the magnetic beads 2 can be prevented from becoming insufficient or excessive.

In the biological material purification method according to the embodiment, the inorganic oxide is preferably a silicon oxide.

According to such a configuration, since the silicon oxide is chemically stable, oxidation and corrosion of the magnetic metal particle 22 can be particularly prevented, and the corrosion resistance of the magnetic bead 2 can be particularly improved. Good nucleic acid adsorptivity can be imparted to the coating layer 24.

In the biological material purification method according to the embodiment, the magnetic metal particle 22 may contain an Fe-based amorphous alloy.

According to such a configuration, since the magnetic permeability of the magnetic metal particles 22 is high and the coercive force is low, a biological material can be purified using the magnetic beads 2 having good separability in magnetic separation and excellent redispersibility.

The magnetic beads 2 according to the embodiment are used in the biological material purification method including the adsorption step S202, the separation step S204, and the elution step S208. In the adsorption step S202, the magnetic beads 2 and the biological material are mixed in the container 1, and the biological material is adsorbed to the magnetic beads 2 in the obtained mixed liquid. In the separation step S204, the magnetic beads 2 to which the biological material is adsorbed are magnetically separated from the mixed liquid in the container 1. In the elution step S208, the biological material is eluted from the magnetic beads 2 into the elution liquid in the container 1.

The magnetic beads 2 contain the magnetic metal particle 22 and the coating layer 24 containing the inorganic oxide and coating the surface of the magnetic metal particle 22.

The particle diameter d31 of the magnetic beads 2 is 1.1 μm or more and 20.4 μm or less, and the surface area when the volume is 7 μL is 12,000 mm² or more and 230,000 mm² or less.

According to such a configuration, the particle diameter d31 and the surface area are optimized, and thus the magnetic beads 2 capable of preventing the loss during the purification process while reducing the time required for adsorbing the biological material can be implemented. Accordingly, even when the amount of the magnetic beads 2 to be used is reduced, an adsorption amount of the biological material can be secured, and the biological material can be purified with a high yield.

By reducing the amount of the magnetic beads 2 to be used, unintentional remaining of the wash solution or the like between the magnetic beads 2 during the purification process (occurrence of poor purification due to the residual liquid) can be prevented. Accordingly, the magnetic beads 2 capable of preventing the inspection failure of the biological material due to the residual liquid can be implemented.

The magnetic bead dispersion liquid 70 according to the embodiment includes the magnetic beads 2 (magnetic bead according to the embodiment) and the dispersion medium 71 in which the magnetic beads 2 are dispersed.

According to such a configuration, the magnetic bead dispersion liquid 70 useful for the biological material purification method described above is obtained.

The biological material purification kit 100 according to the embodiment includes the magnetic bead dispersion liquid 70 (magnetic bead dispersion liquid according to the embodiment).

According to such a configuration, the biological material purification kit 100 useful for the biological material purification method described above is obtained.

Although the biological material purification method, the magnetic bead, the magnetic bead dispersion liquid, and the biological material purification kit according to the present disclosure have been described above based on the illustrated embodiments, the present disclosure is not limited thereto. For example, the biological material purification method according to the present disclosure may be a method in which a step for any purpose is added to the embodiment. In each of the magnetic bead, the magnetic bead dispersion liquid, and the biological material purification kit according to the present disclosure, any configuration may be added to the embodiment, or a part of the configuration of the embodiment may be replaced with another configuration having the same function.

### EXAMPLE

Next, specific examples of the present disclosure will be described.

### 5. PRODUCTION OF MAGNETIC BEADS AND NUCLEIC ACID PURIFICATION

Magnetic beads in Examples and Comparative Examples were produced as follows, and then, nucleic acid purification was performed using the produced magnetic beads.

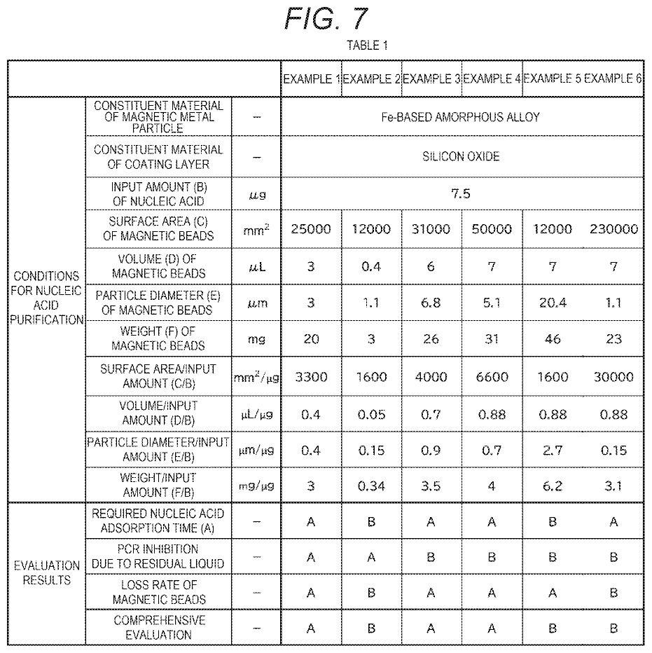

FIG. 7 is Table 1 illustrating conditions for nucleic acid purification using the magnetic beads in Examples and evaluation results of nucleic acid purification.

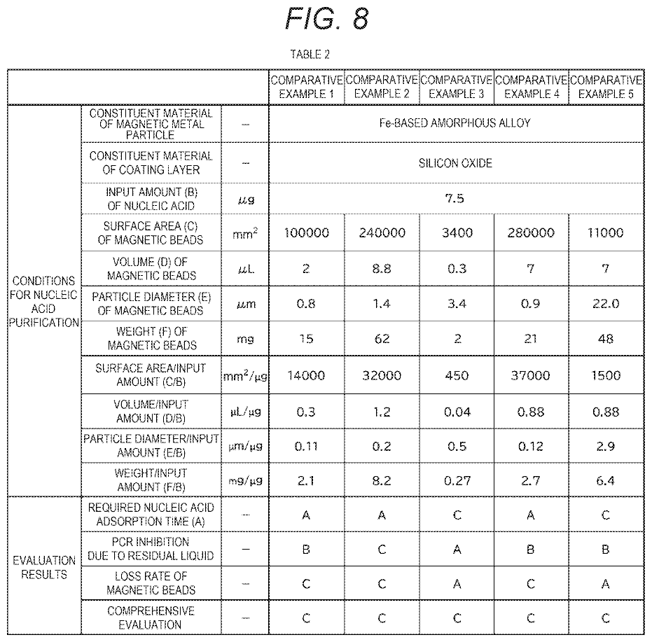

FIG. 8 is Table 2 illustrating conditions for nucleic acid purification using the magnetic beads in Comparative Examples and evaluation results of nucleic acid purification.

### 5.1. Example 1

First, the production of magnetic beads and nucleic acid purification of Example 1 will be described.

### 5.1.1. Preparation of Magnetic Beads

First, an Fe-based amorphous alloy powder produced by a water atomization method was prepared as magnetic metal particles.

Next, tetraethoxysilane (TEOS) was brought into contact with the surface of the magnetic metal particles by a Stöber method, and then firing was performed to form a coating layer composed of a silicon oxide. Accordingly, magnetic beads were obtained.

### 5.1.2. Nucleic Acid Purification

First, the magnetic bead dispersion liquid containing the magnetic beads of Example 1 was taken into a 1.5 mL tube and left to stand until room temperature was reached. The particle diameter d31 (E) of the used magnetic beads is as shown in Table 1 (FIG. 7). An amount of the magnetic beads taken into the tubes was adjusted so as to satisfy the surface area (C) of the magnetic beads, the volume (D) of the magnetic beads, and the weight (F) of the magnetic beads shown in Table 1.

Next, another 1.5 mL tube was prepared, and a sample liquid containing a nucleic acid was taken into the tube. At this time, an amount of the sample liquid was adjusted such that an amount of the nucleic acid in the tube was 7.5 μg. The sample liquid was prepared in advance by preparing a nucleic acid (Salmon Sperm) with a Buffer AVL buffer solution so as to have a concentration of 3.75 [mg/mL], and left to stand until room temperature was reached.

Next, an adsorption step was performed. Specifically, 750 μL of the magnetic bead dispersion liquid and lysis-adsorption liquid were added to a tube into which the sample liquid was charged. Then, the substances accommodated in the tube were stirred for 9 min with a vortex mixer.

Next, a separation step was performed. Specifically, the tube after the stirring was set in a magnetic stand, and a magnetic separation treatment and a liquid discharging treatment were performed.

Next, a washing step was performed. Specifically, 900 μL of a wash solution adjusted to room temperature was added to the tube after the separation step. An aqueous solution containing 7.3 M of guanidine hydrochloride was used as the wash solution. Then, the substances accommodated in the tube were stirred for 5 sec with a vortex mixer. Thereafter, the magnetic separation treatment was performed. In the magnetic separation treatment, the tube was set on a magnetic stand and left for 20 sec. Subsequently, the liquid discharging treatment was performed to remove the wash solution in the supernatant. The above treatment was defined as a first cleaning treatment, and this treatment was performed again. Subsequently, 900 μL of a wash solution adjusted to room temperature was added to the tube after the first cleaning treatment. As the wash solution, 70 v/v % ethanol obtained by adding 70 mL of ethanol to 30 mL of water was used. Then, the substances accommodated in the tube were stirred for 5 sec with a vortex mixer. Thereafter, the magnetic separation treatment was performed. In the magnetic separation treatment, the tube was set on a magnetic stand and left for 20 sec. Subsequently, the liquid discharging treatment was performed to remove the wash solution in the supernatant. The above treatment was defined as a second cleaning treatment, and this treatment was performed again.

Next, the elution step was performed. Specifically, 100 μL of an elution liquid adjusted to room temperature was added to the tube after the washing step. Pure water was used as the elution liquid. Then, the substances accommodated in the tube were stirred for 10 min with a vortex mixer. Accordingly, the nucleic acid captured by the magnetic beads was eluted. Thereafter, the magnetic separation treatment was performed. In the magnetic separation treatment, the tube was set on a magnetic stand and left for 30 sec. Subsequently, a liquid discharging treatment was performed, and the elution liquid in the supernatant was collected.

Table 1 shows, as conditions for the nucleic acid purification, a configuration of the used magnetic beads, the input amount (B) of the nucleic acid, and the ratios of the surface area (C), the volume (D), the particle diameter (E), and the weight (F) of the magnetic beads to the input amount (B) of the nucleic acid.

Other characteristics of the used magnetic beads are as follows.
Average particle diameter D50: 10 μm
Saturation magnetization: 110 emu/g
Coercive force: 50 A/m
Thickness of coating layer: 30 nm

### 5.2. Examples 2 to 6 and Comparative Examples 1 to 5

Magnetic beads were prepared and nucleic acid purification was performed in the same manner as in Example 1 except that a configuration of magnetic beads and conditions for nucleic acid purification were changed as shown in Table 1 (FIG. 7) or Table 2 (FIG. 8).

### 6. EVALUATION OF NUCLEIC ACID PURIFICATION

Next, results of nucleic acid purification in Examples and Comparative Examples were evaluated.

### 6.1. Required Nucleic Acid Adsorption Time (A)

In the adsorption step, a time for stirring substances accommodated in a tube was defined as a “nucleic acid adsorption time”. Then, the above nucleic acid purification was performed while changing the nucleic acid adsorption time in increments of 1 min, and a nucleic acid elution amount was determined. The nucleic acid elution amount was determined as follows.

First, the collected elution liquid was charged into a 1.5 mL tube, and 80 μL of the elution liquid was taken out therefrom and charged into a spectroscopic cell. Next, 240 μL of pure water was added to the spectroscopic cell to prepare a sample.

Next, the spectroscopic cell containing the sample was set in a cell holder of a spectrophotometer. As the spectrophotometer, a NanoDrop microspectrophotometer manufactured by Thermo Fisher Scientific was used. Then, a concentration of the nucleic acid in the elution liquid was measured with the spectrophotometer.

Next, the nucleic acid elution amount was calculated using the measured nucleic acid concentration and the liquid amount of the elution liquid.

Next, the nucleic acid yield was calculated using the calculated nucleic acid elution amount and the input nucleic acid weight.

Next, the nucleic acid adsorption time required for the nucleic acid yield to exceed 10% was determined as the “required nucleic acid adsorption time (A)”. Then, the obtained required nucleic acid adsorption time was evaluated according to the following evaluation criteria.
A: The required nucleic acid adsorption time (A) is less than 1 min.
B: The required nucleic acid adsorption time (A) is 1 min or longer and less than 9 min.
C: The required nucleic acid adsorption time (A) is 9 min or longer

### 6.2. PCR Inhibition Due to Residual Liquid

First, 2 μL of the collected elution liquid was supplied to a 10 μL real-time PCR reaction system to determine the number of cycles until the PCR amplification curve rose. When the wash solution or the like is absorbed into the magnetic beads and becomes a residual liquid, the residual liquid may transfer to the elution liquid and cause PCR inhibition. At this time, the number of cycles increases. Therefore, PCR inhibition due to the residual liquid was evaluated by observing, according to the following evaluation criteria, the number of cycles until the PCR amplification curve rose. The evaluation results are shown in Tables 1 and 2.
A: PCR amplification curve rose within 30 cycles
B: PCR amplification curve rose after 30 cycles
C: PCR amplification curve does not rise

### 6.3. Loss Rate of Magnetic Beads

First, the wash solution discharged in the washing step was charged in a spectroscopic cell and set in a cell holder of a spectrophotometer. Then, a magnetic bead concentration of the wash solution was measured with the spectrophotometer. In this measurement, a calibration curve prepared from a sample having a known magnetic bead concentration was used.

Next, a loss rate of the magnetic beads was calculated based on the measured magnetic bead concentration of the wash solution, the collected wash solution amount, and the input amount of the magnetic beads. Then, the calculated loss rate of the magnetic beads was evaluated in view of the following evaluation criteria. The evaluation results are shown in Tables 1 and 2.
A: The loss rate of the magnetic beads is less than 1%
B: The loss rate of the magnetic beads is 1% or more and less than 15%
C: The loss rate of the magnetic beads is 15% or more

### 6.4. Comprehensive Evaluation

The comprehensive evaluation of the nucleic acid purification was performed by observing the results of the above three evaluations according to the following evaluation criteria. The evaluation results are shown in Tables 1 and 2.
A: The number of evaluation values B is one or less, and the remaining evaluation values are all evaluation values A
B: The number of evaluation values B is two or more, and the evaluation value C is not included
C: One or more evaluation values C are included

From the evaluation results illustrated in Tables 1 and 2, the following can be recognized.

In the nucleic acid purification in Examples, by optimizing the particle diameter and surface area of the magnetic beads, the loss rate of the magnetic beads could be reduced while reducing the required nucleic acid adsorption time (A). Accordingly, it was found that even when the amount of the magnetic beads to be used was reduced, an adsorption amount of the biological material could be secured, and the biological material could be purified with a high yield.

In the nucleic acid purification in Examples, poor purification due to residual liquid being brought in could be prevented, and thus PCR inhibition could be prevented. Accordingly, it was found that the inspection failure of the biological material can be prevented.

It was found that since the particle diameter and the surface area of the magnetic beads in Examples were optimized, significant effects as described above were exhibited when the magnetic beads were used for the nucleic acid purification in Examples.

What is claimed is:

1. A biological material purification method, which is a method for purifying a biological material, the method comprising:
an adsorption step of mixing magnetic beads and the biological material in a container and adsorbing the biological material to the magnetic beads in the obtained mixed liquid;
a separation step of magnetically separating, from the mixed liquid in the container, the magnetic beads to which the biological material is adsorbed; and
an elution step of eluting the biological material from the magnetic beads into an elution liquid in the container, wherein
the magnetic beads contain a magnetic metal particle and a coating layer containing an inorganic oxide and coating a surface of the magnetic metal particle,
the magnetic beads in the container have a particle diameter d31 of 1.1 μm or more and 20.4 μm or less, and
the magnetic beads in the container have a surface area of 12,000 mm² or more and 230,000 mm² or less.

2. The biological material purification method according to claim 1, wherein
the biological material is a nucleic acid.

3. The biological material purification method according to claim 2, wherein
the following formula (1) is satisfied,
$$A < 9. [ min ] \qquad ( 1 )$$
where B is an input amount [μg] of the biological material in the container, X is an elution amount [μg] of the biological material eluted from the magnetic beads in the elution step, and A is a required adsorption time [min] for the biological material until a ratio X/B of the elution amount X to the input amount B exceeds 10%.

4. The biological material purification method according to claim 2, wherein
in the adsorption step, the following formula (2) is satisfied,
$$1 , 500 [ {mm}^{2} / μ g ] \leq C / B \leq 30 , 000 [ {mm}^{2} / μ g ] \qquad ( 2 )$$
where B is an input amount [μg] of the biological material in the container and C is the surface area [mm²] of the magnetic beads in the container.

5. The biological material purification method according to claim 2, wherein
in the adsorption step, the following formula (3) is satisfied,
$$0.03 [ μ L / μg ] \leq D / B < 0.9 [ μL / μg ] \qquad ( 3 )$$
where B is an input amount [μg] of the biological material in the container and D is a volume [μL] of the magnetic beads in the container.

6. The biological material purification method according to claim 2, wherein
in the adsorption step, the following formula (4) is satisfied,
$$0.13 [ μm / μg ] \leq E / B < 2.8 [ μm / μg ] \qquad ( 4 )$$
where B is an input amount [μg] of the biological material in the container and E is the particle diameter d31 [μm] of the magnetic beads in the container.

7. The biological material purification method according to claim 2, wherein
in the adsorption step, the following formula (5) is satisfied,
$$0.3 [ mg / μg ] \leq F / B < 6.5 [ mg / μg ] \qquad ( 5 )$$
where B is an input amount [μg] of the biological material in the container and F is a weight [mg] of the magnetic beads in the container.

8. The biological material purification method according to claim 1, wherein
the inorganic oxide is a silicon oxide.

9. The biological material purification method according to claim 1, wherein
the magnetic metal particle contains an Fe-based amorphous alloy.

10. A magnetic bead, which is a magnetic bead to be used in a biological material purification method, the method including an adsorption step of mixing the magnetic bead and a biological material in a container and adsorbing the biological material to the magnetic bead in the obtained mixed liquid, a separation step of magnetically separating, from the mixed liquid in the container, the magnetic bead to which the biological material is adsorbed, and an elution step of eluting the biological material from the magnetic bead into an elution liquid in the container, the magnetic bead comprising:
a magnetic metal particle; and
a coating layer containing an inorganic oxide and coating a surface of the magnetic metal particle, wherein
a particle diameter d31 is 1.1 μm or more and 20.4 μm or less, and
a surface area when a volume is 7 μL is 12,000 mm² or more and 230,000 mm² or less.

11. A magnetic bead dispersion liquid comprising:
the magnetic bead according to claim 10; and
a dispersion medium in which the magnetic bead is dispersed.

12. A biological material purification kit comprising:
the magnetic bead dispersion liquid according to claim 11.
